(19) United States
(12) Patent Application Publication
(10) Pub. No.: US 2022/0233598 A1
(43) Pub. Date: Jul. 28, 2022

(54) METHODS OF PREVENTATIVE THERAPY FOR POST-TRAUMATIC OSTEOARTHRITIS

(71) Applicant: UNIVERSITY OF IOWA RESEARCH FOUNDATION, Iowa City, IA (US)

(72) Inventors: Kyungsup Shin, Iowa City, IA (US); Dong Rim Seol, Iowa City, IA (US); James A. Martin, Iowa City, IA (US); Ino Song, Iowa City, IA (US)

(73) Assignee: UNIVERSITY OF IOWA RESEARCH FOUNDATION, Iowa City, IA (US)

(21) Appl. No.: 17/585,094

(22) Filed: Jan. 26, 2022

Related U.S. Application Data
(60) Provisional application No. 63/142,451, filed on Jan. 27, 2021.

(51) Int. Cl. A61K 35/28; A61K 9/00; A61P 19/02; A61K 47/69

(52) U.S. Cl. CPC A61K 35/28; A61K 47/6903; A61P 19/02; A61K 9/0019

(57) ABSTRACT

In certain embodiments, the present invention provides a method for preventing or treating post-traumatic osteoarthritis (PTOA) in the temporomandibular joint (TMJ) in a subject in need thereof, comprising administrating a pharmaceutical composition comprising an effective amount of mesenchymal stem cell-derived exosomes or mesenchymal stem cell-derived exosomal microRNA to the subject. In certain embodiments, the present invention provides a method for preventing progressive fibrocartilage degeneration in a subject in need thereof, comprising administrating a pharmaceutical composition comprising an effective amount of mesenchymal stem cell-derived exosomes or mesenchymal stem cell-derived exosomal microRNA to the subject.

### CROSS-REFERENCE TO RELATED APPLICATION

This application claims priority to U.S. Provisional Application No. 63/142,451 that was filed on Jan. 27, 2021. The entire content of the applications referenced above is hereby incorporated by reference herein.

### STATEMENT REGARDING FEDERALLY SPONSORED RESEARCH

This invention was made with government support under DE030166 awarded by National Institutes of Health. The government has certain rights in the invention.

### BACKGROUND

The temporomandibular joint (TMJ) is a ginglymoarthrodial synovial joint that is composed of two joints connecting the lower jaw and the skull with the intermediate articular disc between the joints. The unique structure of the TMJ allows the movement of the jaw while being exposed to various mechanical stimulations during daily activities. Although the fibrocartilagenous temporomandibular joint (TMJ) has evolved to facilitate mastication, disorders that lead to restricted motion, jaw pain, and malocclusion are common. Temporomandibular disorders (TMDs) of various etiologies associated with gender, trauma, and psychological factors, are the second most prevalent musculoskeletal conditions. In particular, TMJ post-traumatic osteoarthritis (PTOA) caused by traumatic injury is an articular degenerative disease with pain and limited mouth opening, which is pathologically manifested as a rapidly progressive loss of articular fibrocartilage. Current treatment options are limited to surgical interventions designed to relieve pain and restore normal joint motion; however, such procedures do not address progressive fibrocartilage degeneration, a root cause of many symptoms. Accordingly, new therapies to treat TMJ PTOA are needed.

### SUMMARY

In a one aspect, provided herein is a method for preventing or treating post-traumatic osteoarthritis (PTOA) in the temporomandibular joint (TMJ) in a subject in need thereof, comprising administrating a pharmaceutical composition comprising an effective amount of mesenchymal stem cell-derived exosomes or mesenchymal stem cell-derived exosomal microRNA to the subject.

In a one aspect, provided herein is a method for preventing progressive fibrocartilage degeneration in a subject in need thereof, comprising administrating a pharmaceutical composition comprising an effective amount of mesenchymal stem cell-derived exosomes or mesenchymal stem cell-derived exosomal microRNA to the subject.

In certain aspects, the pharmaceutical composition is a local delivery system.

In certain aspects, the local delivery system comprises an injectable temperature-sensitive hydrogel.

In certain aspects, the local delivery system comprises an engineering exosome vehicle.

In certain aspects, the pharmaceutical composition is administered by injection to the subject.

In certain aspects, the pharmaceutical composition comprising an effective amount of mesenchymal stem cell-derived exosomes.

In certain aspects, the pharmaceutical composition comprising an effective amount of mesenchymal stem cell-derived exosomal microRNA.

In certain aspects, the mesenchymal stem cell-derived exosomes or mesenchymal stem cell-derived exosomal microRNA are bone marrow stem cell-derived exosomes (BMSC-Exo).

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A-1E. Characteristics of SBMSC-Exo. (FIG. 1A) Schematic diagram of exosome isolation. (FIG. 1B and FIG. 1C) Concentration and size distribution of SBMSC-Exo by MRPS (n=4). (FIG. 1B) The box and whisker plot with median values (horizontal line), 25^{th} and 75^{th} percentiles (lower and upper box limits), and maximum and minimum data (vertical error bars). (FIG. 1C) A representative graph of SBMSC-Exo size distribution. (FIG. 1D) Morphology of SBMSC-Exo by TEM. Bar=200 nm. (FIG. 1E) Immunoblotting assay by Exo-Check antibody array. A negative marker: GM130 (cis-Golgi matrix protein), positive markers: FLOT-1 (Flotillin-1), ICAM (intercellular adhesion molecule), ANXA5 (annexin A5), and TSG101 (tumor susceptibility gene 101).

FIGS. 2A-2I. Internalization of SBMSC-Exo into FCSCs and chemotactic effect of SBMSC-Exo. (FIG. 2A) A custom drop-tower device with 2 mm-diameter impactor. (FIG. 2B and FIG. 2C) Confocal images of migratory FCSCs into an impact-injured superficial layer of mandibular condyle at day 1 (FIG. 2B) and 7 (FIG. 2C). (FIGS. 2D-2G) in vitro internalization of PKH-67-stained (green) SBMSC-Exo (FIG. 2E and FIG. 2G) and PBS control (FIG. 2D and FIG. 2F) into FCSCs. (FIG. 2D and FIG. 2E) No counterstaining. (FIG. 2F and FIG. 2G) DAPI (blue) counterstaining. (FIG. 2H) Cytotoxicity of SBMSC-Exo)(1×10⁸⁻¹⁰ to FCSC at 48 hours in serum-free CM (n=5). (FIG. 2I) In vitro chemotactic effect of SBMSC-Exo (1×10⁹ or 1×10¹⁰) (n=5). All scale bars=100 μm, *p<0.05, **p<0.01.

FIGS. 3A-3B. FCSC cytotoxicity under oxidative stress and SBMSC-Exo treatment. FCSCs were cultured in culture medium (CM) with 2% exosome-depleted FBS. (FIG. 3A) Effect of 150 μM H₂O₂ treatment for 3 hours on FCSC viability at 24 hours (n=5). NS: not significant. (FIG. 3B) Effect of SBMSC-Exo on FCSC viability at 24 hours (n=5).

FIGS. 4A-4C. Protective effect of SBMSC-Exo on ROS accumulation by DHE assay. FCSCs were treated with 150 μM H₂O₂ for 3 hours followed by the co-culture with or without SBMSC-Exo for 24 hours. (FIG. 4A) Confocal images of FCSCs stained with DHE (green: Calcein AM, red: DHE, scale bars=100 μm). (FIG. 4B) Calculated ROS relative fluorescence unit (RFU) from confocal images and then normalized by the mean of positive control (H₂O₂⁺/SBMSC-Exo⁻) (n=3). (FIG. 4C) ROS measurement by a fluorescence-based quantification assay (n=5, *p<0.05, **p<0.01).

FIGS. 5A-5C. Protective effect of SBMSC-Exo on ROS accumulation by DHE assay. FCSCs were treated with 150 μM H₂O₂ for 3 hours followed by the co-culture with or without SBMSC-Exo for 24 hours. (FIG. 5A) Confocal images of FCSCs stained with Carboxy-H₂DCFDA (green) (scale bars=100 μm). (FIG. 5B) Calculated ROS relative fluorescence unit (RFU) from confocal images and then normalized by the mean of positive control (H₂O₂⁺/SBMSC-Exo⁻) (n=3). (FIG. 5C) ROS measurement by a fluorescence-based quantification assay (n=5, *p<0.05, **p<0.01).

FIGS. 6A-6B. Therapeutic strategy of exome for preventing post-traumatic osteoarthritis (PTOA) in the temporomandibular joint (TMJ). (FIG. 6A) Prevention of TMJ PTOA using subchondral bone-derived mesenchymal stem cell-derived exosome (SBMSC-Exo). In damaged fibrocartilage with oxidative stress, SBMSC-Exo simulates fibrocartilage stem cells (FCSCs) migration and alleviates reactive oxygen species (ROS) accumulation by delivering antioxidant cargoes and forming chemokine gradients. (FIG. 6B) Prevention of TMJ PTOA using synthesized miroRNAs (miRNAs) and engineering exosome vehicles. Synthesized miRNAs related to chemotaxis and antioxidation are selected via New Generation Sequencing (NGS) and loaded in engineered exosome for effective delivery. This approach avoids tissue harvest and ex vivo cell expansion for exosome isolation.

FIG. 7. Overall strategy of exosome-base preventative therapy for post-traumatic osteoarthritis (PTOA).

FIGS. 8A-8F. Characterization of BMSC-Exo. (FIG. 8A) A graph of BMSC-Exo size distribution. (FIG. 8B) Zeta-potential of BMSC-Exo. (FIG. 8C) The expression of exosomal protein markers. (FIG. 8D) Morphology of BMSC-Exo by TEM (scale bar=200 nm). (FIG. 8E and FIG. 8F) In-vitro internalization of PKH-67 stained BMSC-Exo (green) (FIG. 8E) and PB control (FIG. 8F) into FCSC (scale bar=100 μM).

FIGS. 9A-9B. Effect of BMSC-Exo on FCSC proliferation and chemotaxis. (FIG. 9A) Cytotoxicity of BMSC-Exo on FCSC at 48 hours (n=5). (FIG. 9B) FCSC proliferation on day 1, 3, and 7 treated with three-concentration of BMSC-Exo from MTS proliferation assay (n=6). *p<0.05, **p<0.01.

FIGS. 10A-10B. FIG. 3. Protective effect of BMSC-Exo on the mitochondrial superoxide accumulation by MitoSox assay. (FIG. 10A) Confocal images of FCSCs stained with MitoSox and MitoTracker green (green: MitoTracker green, red: MitoSox, scale bars=100 μm). (FIG. 10B) Calculated superoxide relative fluorescence unit (RFU) fssssrom confocal images (n=6, *p<0.05).

FIGS. 11A-11D. Characteristics of BMSC-Exo. (FIG. 11A) Size distribution of BMSC-Exo by MRPS (n=4). (FIG. 11B) Morphology of BMSC-Exo by TEM. Bar=200 nm. (FIG. 11C) Zeta Potentials of BMSC-Exo (n=3) (FIG. 11D) Immunoblotting assay by Exo-Check antibody array.

FIGS. 12A-12D. Therapeutic effects of BMSC-Exo. (FIG. 11A) Proliferation of FCSC by MTS assay. (FIG. 11B) Chemotactic migration of FCSC by Transwell Assay. (FIG. 11C) Confocal images of FCSCs stained with MitoSox (green) and Hoechst 33342 (blue) (scale bars=100 μm). (FIG. 11D) Relative fluorescence unit (RFU) from confocal images (normalized by NTC).

### DETAILED DESCRIPTION

Extracellular vesicles are important mediators of intercellular communication, which not only participate in normal physiological functions, but also affect the occurrence and development of diseases. Exosomes are a type of extracellular vesicle with a diameter of 40 to 100 nanometers (nm), and can be separated by centrifugation from all types of body fluids, including blood, urine, bronchoalveolar lavage fluid, breast milk, amniotic fluid, synovial fluid, pleural exudate, ascites, etc.

Exosomes, which are the extracellular vesicles released from eukaryotic cells, have diagnostic and therapeutic potential without the risks of immune response and tumorigenesis. In one embodiment, bone marrow stem cells (BMSC-Exos) play a pivotal role in protecting oxidative-related injuries and increase the regenerative capacity.

The present disclosure provides a pharmaceutical composition comprising exosomes or exosomal microRNA derived from mesenchymal stem cells, and an injectable hydrogel or engineered exosome vehicle for preventing post-traumatic osteoarthritis (PTOA) in the temporomandibular joint (TMJ).

Exosomes are isolated from the culture medium of mesenchymal stem cells such as bone marrow stem cell, adipose stem cell, umbilical cord stem cell, hematopoietic stem cell, embryonic stem cell, and induced pluripotent stem cell. MicroRNAs are short non-coding RNA molecules in exosomes and bind to target mRNA to regulate gene expression posttranscriptionally. The exosomes or exosomal microRNAs play in alleviating oxidative stress from traumatic injuries in TMJ, eventually prohibiting PTOA. The efficiency of exosomes or exosomal microRNAs can be enhanced in hydrogel or engineered exosome vehicle that is locally delivered into target TMJ, gelled at body temperature, and allows sustained release of exosome. Hydrogel is a three-dimensional network of hydrophilic polymers that can swell in water and hold a large amount of water while maintaining the structure. MicroRNA can be synthesized to avoid stem cell culture as a means of exosome production. Synthetic miRNAs will be loaded in engineered exosomes by electroporation or sonication method, thereby replacing BMSC culture as a potential means of exosome production.

Formulations and Methods of Administration

In certain embodiments, an effective amount of the therapeutic composition is administered to the subject. “Effective amount” or “therapeutically effective amount” are used interchangeably herein, and refer to an amount of a compound, formulation, material, or composition, as described herein effective to achieve a particular biological result.

In certain embodiments, an amount of the therapeutic composition is administered in order to treat to the subject.

In certain embodiments, the therapeutic composition is administered via intramuscular, intradermal, or subcutaneous delivery. In certain embodiments, the therapeutic composition is administered via a mucosal surface, such as an oral, or intranasal surface. In certain embodiments, the therapeutic composition is administered via intrasternal injection, or by using infusion techniques.

In certain embodiments, “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The compositions of the invention may be formulated as pharmaceutical compositions and administered to a mammalian host, such as a human patient, in a variety of forms adapted to the chosen route of administration, i.e., orally, intranasally, intradermally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Additional ingredients such as fragrances or antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

To treat a subject, the therapeutic composition is administered parenterally, usually by intramuscular or subcutaneous injection in an appropriate vehicle. Other modes of administration, however, such as oral delivery or intranasal delivery, are also acceptable. Therapeutic composition formulations will contain an effective amount of the active ingredient in a vehicle.

Formulations will contain an effective amount of the active ingredient in a vehicle, the effective amount being readily determined by one skilled in the art. “Effective amount” is meant to indicate the quantity of a compound necessary or sufficient to realize a desired biologic effect. The active ingredient may typically range from about 1% to about 95% (w/w) of the composition, or even higher or lower if appropriate. The amount for any particular application can vary depending on such factors as the severity of the condition. The quantity to be administered depends upon factors such as the age, weight and physical condition of the animal considered for vaccination and kind of concurrent treatment, if anyTypically, dosages used in vitro may provide useful guidance in the amounts useful for in situ administration of the composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Reminpton's Pharmaceutical Sciences, 17th ed., Mack Publishing Co., Easton, Pa., 1990, each of which is herein incorporated by reference. Additionally, effective dosages can be readily established by one of ordinary skill in the art through routine trials establishing dose response curves. The subject is treated by administration of the composition thereof in one or more doses. Multiple doses may be administered as is required. For example, the initial dose may be followed up with a second dosage after a period of about four weeks. Further dosages may also be administered. The composition may be administered multiple (e.g., 2, 3, 4 or 5) times at an interval of, e.g., about 1, 2, 3, 4, 5, 6 or 7, 14, or 21 days apart.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the present compositions may be combined with one or more excipients and used in the form of ingestible tablets, buccal tablets, troches, capsules, elixirs, suspensions, syrups, wafers, and the like. Such preparations should contain at least 0.1% of the present composition. The percentage of the compositions may, of course, be varied and may conveniently be between about 2 to about 60% of the weight of a given unit dosage form. The amount of present composition in such therapeutically useful preparations is such that an effective dosage level will be obtained.

Useful dosages of the compositions of the present invention can be determined by comparing their in vitro activity, and in vivo activity in animal models. The amount of the compositions described herein required for use in treatment will vary with the route of administration and the age and condition of the subject and will be ultimately at the discretion of the attendant veterinarian or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will now be illustrated by the following non-limiting Examples.

### Example 1

Therapeutic Potential of Exosome in Post-Traumatic Osteoarthritis of the Temporomandibular Joint

Temporomandibular joint (TMJ) post-traumatic osteoarthritis (PTOA) is a degenerative disease of mandibular fibrocartilage that develops secondary to acute injury and joint overuse. It involves various symptoms including pain, joint inflammation, loss of mandibular movement, and abnormal bone remodeling. Oxidative stress is one of the major factors that induces homeostatic imbalance and increases the risk of PTOA. Excessive levels of intercellular reactive oxygen species (ROS) can change cellular functions, including cell growth, migration, and death, and extracellular matrix homeostatis. Exosomes, which are the extracellular vesicles released from eukariotic cells, have been widely studied due to their diagnostic and therapeutic potential. The contents of the exosomes reflect the status of parental cells, thereby, stem cell-derived exosomes deliver various cargoes mimicking the therapeutic potential of the stem cell therapy, while avoiding the risks of immune response and tumorigenesis. A series of prior studies have shown exosomes derived from bone marrow stem cells (BMSC-Exos) increase regenerative and protective capacity of the recipient cells and alleviate oxidative stress.

The present study evaluated the antioxidant and regenerative effects of BMSC-Exos on fibrocartilage stem cells (FCSCs) that play a major role in repair and regeneration of the damaged mandibular fibrocartilage. The BMSCs and FCSCs were collected from the male bovine femur and the superficial layer of the mandibular condyle, respectively. BMSC-Exos isolated from a size exclusion chromatography method were characterized in terms of the size distribution, zeta potential, morphology, exosomal marker proteins, and uptake by FCSC. FCSC migration and proliferation were investigated by chemotaxis and MTS assays, respectively. Oxidative stress was induced by hydrogen peroxide, and ROS levels were measured using flurogenic dyes, MitoSox and MitoTracker Green (as a control).

The results indicated that BMSC-Exos provided chemoattractic stimulation to FCSCs and increased FCSC migration by 5.5-14.8 times in a dose-dependent manner. FCSC proliferation was also dose-dependently increased by up to 33% at 7 days compared to that of non-treated control. Lastly, the hydrogen peroxide-induced ROS accumulation in FCSCs was alleviated by BMSC-Exos by 60%. These results indicate that BMSC-Exos can play a pivotal role in protecting oxidative-related injuries and increase the regenerative capacity of FCSCs. The application of BMSC-Exos in damaged TMJ mandibular condyle has the potential to prevent the progression of TMJ PTOA while enhancing joint repair.

### Example 2

Antioxidant and Chemotactic Effects of Subchondral Bone Mesenchymal Stem Cell-Derived Exosomes on Fibrocartilage Repair in Temporomandibular Joint

Abstract

Background

Although the fibrocartilagenous temporomandibular joint (TMJ) has evolved to facilitate mastication, disorders that lead to restricted motion, jaw pain, and malocclusion are common. Current treatment options are limited to surgical interventions designed to relieve pain and restore normal joint motion; however such procedures do not address progressive fibrocartilage degeneration, a root cause of many symptoms. Yet, recent findings suggest that temporomandibular fibrocartilage harbors fibrocartilage stem cells that are capable of stopping or slowing degeneration.

Methods

To exploit this potential, responses were investigated of fibrocartilage to mesenchymal stem cell-derived exosomes, which have been shown to promote tissue repair in a variety of contexts. It was hypothesized that exosomes would promote chemotaxis of fibrocartilage stem cells to damaged fibrocartilage, a crucial step in the regenerative process, as well as protect cells from oxidative stress, a primary driver of degeneration. Exosomes secreted by cultured subchondral bone-derived mesenchymal stem cells were isolated from a culture medium by size exclusion chromatography and characterized with respect to size distribution and concentration, shape, and exosome-specific protein content. Effects on cell chemotaxis and resistance to oxidative stress were tested in in vitro assays.

Results

It was found that exosomes were readily taken up by fibrocartilage stem cells and stimulated chemotaxis by up to a 15-fold increase over that of untreated controls. The same exosome doses significantly lowered signs of oxidative stress in the cells challenged with hydrogen peroxide.

Conclusions

These findings confirm that stem cell-derived exosomes exert beneficial anti-degenerative effects on fibrocartilage and may have therapeutic value in the treatment of temporomandibular disorders.

### Background

The temporomandibular joint (TMJ) is a ginglymoarthrodial synovial joint that is composed of two joints connecting the lower jaw and the skull with the intermediate articular disc in the middle of the joints. The unique structure of the TMJ has developed in such a way that it facilitates the movement of the jaw while being exposed to various mechanical stimulations, including compression, tension, shear, and hydrostatic pressure during daily activities. Meanwhile, temporomandibular disorders (TMD) of various etiologies associated with gender, trauma, and psychological factors, are the second most prevalent musculoskeletal conditions after chronic low back pain. Epidemiologic studies revealed that about 40% of the general population shows at least one TMD symptom, such as sounds and pain, masticatory muscle pain, or restricted mouth opening, and it is highly associated with oral health-related quality of daily life. In addition, according to the National Institutes of Health (NIH), the estimated annual cost for TMJ management was $4 billion in the United States in 2018. Although symptoms are usually temporary and can be managed by conservative treatments, at least 15% of TMD cases become chronic and require either treatments, such as hyaluronic acid injection, or surgical interventions, such as arthrocentesis, arthroplasty, condylotomy, or total joint replacement.

In order to repair TMD, stem/progenitor cell transplantation is considered one of the most promising biological procedures because of the benefits of delaying or avoiding a surgical procedure while restoring TMJ function. Numerous studies have shown the efficacy of TMJ repair and regeneration using the differentiation potential of either allogenic or autogenic stem/progenitor cells. However, the risks associated with the cell transplantation, such as immunological reactions or disease transmission, and the unpredictable long-term behavior of the cells, including the potential of tumor formation, are the major hindrances in bringing cell transplantation to clinical application. In this regard, endogenous stem/progenitor cell homing via a chemokine gradient with minimally invasive treatment has been suggested as a considerable alternative for in situ tissue repair/regeneration. In particular, fibrocartilage stem cells (FCSCs) reside underneath the superficial layer of mandibular condyle fibrocartilage and play a critical role in maintaining homeostatic equilibrium. For instance, one group found that the FCSC pool and the homeostasis of fibrocartilage are regulated via a Wnt signaling system, and an exogenous Wnt inhibitor (SOST) repaired damaged mandibular fibrocartilage in a rabbit model. Likewise, another group identified the existence of endogenous FCSC in human mandibular condylar fibrocartilage, and SOX9 plays a regulatory role in chondrogenic differentiation of human FCSC. Thus, exogenously-induced FCSC homing offers considerable advantages as a therapeutic strategy for TMD.

Oxidative stress is a major factor that induces homeostatic imbalance and increases the risk of TMD. Under normal conditions, reactive oxygen species (ROS). including hydrogen peroxide (H₂O₂) and superoxide anion (O₂·⁻), have roles in general cell metabolism and signal transduction. The level of ROS is controlled by three major mechanisms: redox signaling, detoxification, and Nrf2/Keap1 signaling. Metabolic harmony among these mechanisms is achieved by various antioxidant proteins, such as TRX, PRX, GSH/GSSG, and essential and non-essential amino acids. Multiple risk factors, such as traumatic injury, aging, mitochondrial dysfunction, and abnormal levels of antioxidant molecules, may induce over-production of ROS in both intra- and extra-cellular spaces. When the level of intercellular ROS exceeds the level of antioxidant capacity of the cells, it modifies cellular functions, including cell growth/death and inflammatory response. In addition, it activates the upregulation of matrix metalloproteinases (MMPs) and inhibits extracellular matrix synthesis and stem/progenitor cell migration, eventually leading to degenerative fibrocartilage and osteoarthritis. Therefore, the enhancement of antioxidant activity by delivering antioxidant-relevant molecules is a novel strategy to both protect FCSCs against oxidative stress and keep their ability to regenerate TMJ fibrocartilage.

Over the last decade, mesenchymal stem cell (MSC)-derived exosomes have attracted great attention for their therapeutic uses for various pathologies. The many therapeutic effects of MSC-derived exosomes, including their critical roles in antioxidant and chemotactic migration of recipient cells, have been studied extensively. For instance, MSC-derived exosomes increase chemotactic migration of cardiac stem cells isolated from the hearts of neonatal Sprague Dawley rats in a dose-dependent manner. Also, adipose-derived MSC-exosomes promoted breast cancer cell migration through Wnt signaling pathway. When it comes to the antioxidant effect, MSC-derived exosomal microRNA (miRNA)-21 protected C-kit⁺ cardiac stem cells against H₂O₂ induced oxidative damage by regulating the PTEN/PI3K/Akt pathway. It was demonstrated that MSC-derived exosomes provided a hepatoprotective effect against CC14-induced fibrosis through antioxidant defenses. Furthermore, it was revealed that MSC-derived exosomes protected the lung against pulmonary arterial hypertension through mitochondrial health improvement by regulating TCA cycle. Even though many preclinical studies have shown the promising potential of MSC-derived exosomes as an antioxidant and chemoattractant, it has not been thoroughly studied in the TMJ.

Therefore, it was studied whether subchondral bone mesenchymal stem cell-derived exosomes (SBMSC-Exo) induce chemotactic migration and protect the FCSCs in mandibular condylar fibrocartilage from oxidative stress. To test this, exosomes were isolated from the subchondral bone-derived mesenchymal stem cell (SBMSC) because their origin and micro-environments are similar to bone and fibrocartilage tissue in the TMJ. Chemotactic migration of FCSCs by the chemokine gradient of SBMSC-Exo and alleviation of the H₂O₂-induced oxidative stress in FCSCs was demonstrated.

### Methods

Subchondral Bone Mesenchymal Stem Cell Isolation

SBMSCs were isolated from the subchondral bone of young male bovine knees (18-24 months old). In brief, the bone marrow in subchondral bone was harvested and centrifuged at 300 g for 5 minutes (min) followed by filtration using a 70 μm cell strainer (Corning, Corning, N.Y., USA). Centrifuged pellet was re-suspended and cultured for 5 days at 37° C. in a 5% O₂ and 5% CO₂ atmosphere in culture medium (CM), which consisted of 10% fetal bovine serum (FBS; Thermo Fisher Scientific, Waltham, Mass.), 1% (v/v) penicillin-streptomycin (PS; 10,000 U/ml penicillin and 10,000 μg/ml streptomycin; Gibco, Carlsbad, Calif., USA), and 1% amphotericin B (AB; Sigma-Aldrich, St. Louis, Mo., USA) in Nutrient Mixture F-12/Dulbecco's Modified Eagle Medium (F12/DMEM; both from Gibco).

SBMSC-Derived Exosome Isolation and Purification

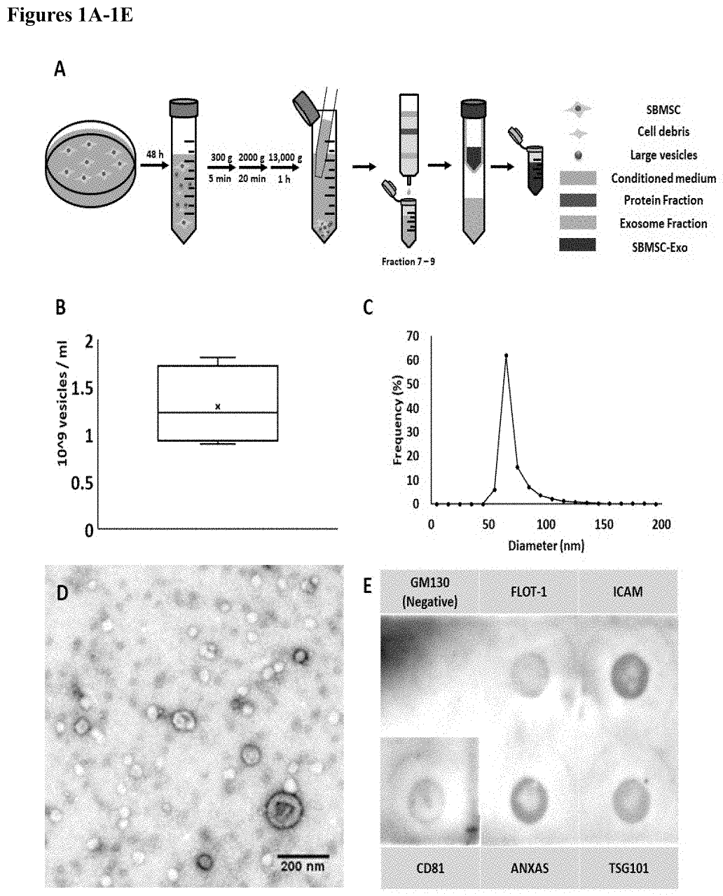

SBMSC-Exo was collected from a SBMSC (passages 2 and 3) conditioned medium (FIG. 1A). Once the cell confluency has reached 80%, a culture plate was washed with serum-free CM in order to remove any FBS-derived exosome and cultured in the exosome culture medium (Exo-CM), which consisted of F12/DMEM with 10% exosome-depleted FBS and 1% PS and AB. After 48 hours, the conditioned medium was collected and pre-processed by the sequential centrifugation at 300 g for 5 min, 2,000 g for 20 min, and 13,000 g for 1 hour without the break in order to remove live cells, cell debris, and large vesicles, respectively. Then the supernatant was collected and concentrated using 10 kDa Amicon Ultra-15 Centrifugal Filter Unit (MilliporeSigma, St. Louis, Mo., USA), and SBMSC-Exo was purified by the size exclusion chromatography (SEC) column (qEVoriginal; Izon Science, Medford, Mass., USA) according to the manufacturer's protocol. Briefly, the SEC column was washed in 15 ml endotoxin-free phosphate buffered saline (PBS) and then 0.5 ml concentrated supernatant was carefully added to the top of the column. The first 6 fractions (0.5 ml each) were discarded, and SBMSC-Exo fractions (fractions 7-9) were collected. Endotoxin-Free PBS was carefully added as needed without making disturbance while collecting the desired fractions. SBMSC-Exo fractions were concentrated again using 10 kDa Amicon Ultra-4 Centrifugal Filter Unit (MilliporeSigma).

Size Measurement and Quantification

Microfluidic resistive pulse sensing (MRPS) method was used to measure the quantity and size of SBMSC-Exo using nCS1 instrument (Spectradyne, Torrance, Calif., USA). In brief, SBMSC-Exo was diluted in PBS with 1% Tween 20, and 5 μl of SBMSC-Exo was added to a TS-300 cartridge, which provided the coverage of 50-300 nm vesicle size, over a range of sample concentration 2×10⁷-5×10¹¹ vesicles/ml. Cartridge was inserted to the instrument, and sample reading process was repeated until standard error of measurement became less than 0.5%.

Immunoblotting Array

Purified SBMSC-Exo was verified using Exo-Check arrays (System Biosciences, Palo Alto, Calif., USA) for purported exosome markers according to the manufacturer's protocol. Briefly, SBMSC-Exo was mixed with a 10×lysis buffer and labeling reagent. An excess labeling reagent was washed using a column filter, and the sample was mixed with a blocking buffer and incubated with a membrane overnight on the shaker at 4° C. The next day, the membrane was washed and incubated with the detecting buffer for 30 min at room temperature. Then, the membrane was scanned using an LAS-4000 (Fujifilm, Tokyo, Japan) followed by membrane development using WesternBright Sirius (Advansta, Menlo Park, Calif., USA) for 2 min.

Transmission Electron Microscopy (TEM)

The morphology of the SBMSC-Exo was visualized in TEM (JEM-1230; JEOL, Peabody, Mass., USA). SBMSC-Exo was placed onto a carbon-coated copper grid (FCF300-Cu, Electron Microscopy Sciences, Hatfield, Pa., USA) for 1 min and counterstained with 1% uranyl acetate for 1 min. Excess UA was removed, and the copper grid was analyzed under TEM.

Ex Vivo FCSC Migration

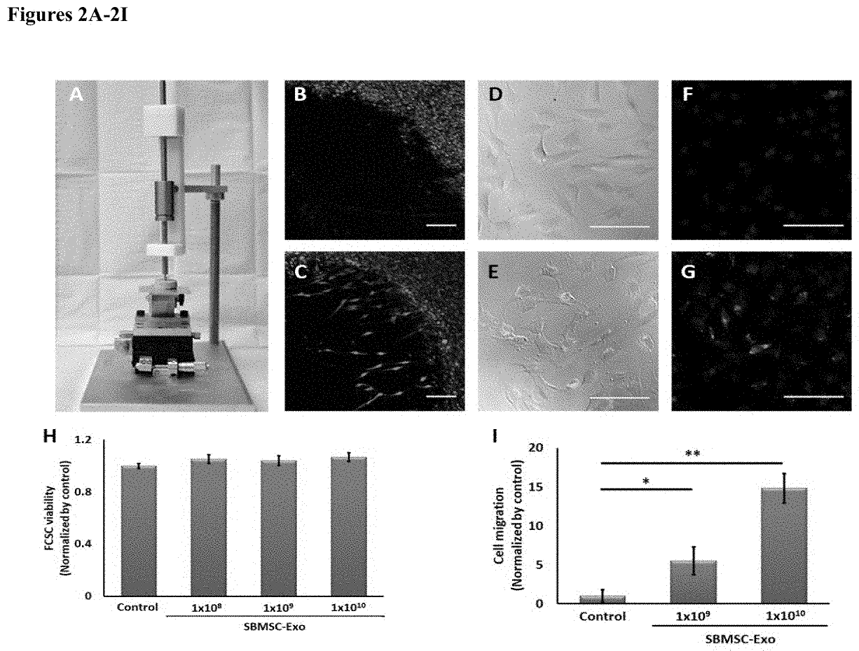

The superficial layer of mandibular condyle was harvested and uniformly dissected using a 6 mm-diameter biopsy punch. Impact loading (40 mJ) was delivered to the middle of the tissue via a custom drop-tower device with a 2 mm-diameter impactor (FIG. 2A). The magnitude of impact energy was chosen because it induced immediate cell death in the area of impact without fibrocartilage tearing. The tissue was washed with Hanks' balanced salt solution (HBSS; Gibco) with 1% PS and AB and cultured in CM. Cell viability and ROS accumulation were confirmed by staining with 1 μg/mL Calcein AM (Thermo Fisher Scientific) for 40 min at day 1 and 7. Green fluorescence (Calcein AM) was visualized by an Olympus FV1000 confocal laser scanning microscope (Olympus Corporation, Tokyo, Japan) within the zone of impact.

Fibrocartilage Stem Cells Isolation

FCSCs were isolated from the superficial layer of mandibular condyle fibrocartilage using a single-cell suspension method. In brief, the layer was carefully harvested and digested using 0.35 μg/mL collagenase and 0.35 μg/mL pronase (both from Sigma-Aldrich) for 4 hours. The mixture of cells and digestion reagents were centrifuged at 300 g for 5 min followed by filtration through a 70 μm cell strainer. Early passages (2^{nd} and 3^{rd}) of FCSCs were used for in vitro studies.

Uptake Assay

Internalization of the SBMSC-Exo into FCSCs was evaluated using a PKH67 Green Fluorescent Cell Linker Mini Kit (Sigma-Aldrich) according to the manufacturer's protocol. In brief, 10 μl of SBMSC-Exo (1.4×10¹⁰ vesicles) was mixed with PKH-67 staining reagent followed by the dilution using diluent C. PBS was used as a negative control. Excessive PKH-67 staining reagent in both SBMSC-Exo and PBS groups was removed by Exosome Spin Columns (MW 3000; Thermo Fisher Scientific). FCSCs (5×10³ cells/well) was seeded in a 16-chamber slide and cultured in CM for 3 days. The cells were washed with serum-free CM and incubated in 2% Exo-CM with PKH-67-stained SBMSC-Exo at 37° C. for 24 hours. For the counterstaining, 4′,6-diamidino-2-phenylindole (DAPI; Life Technologies, Gaithersburg, Md., USA) was used with 30 min incubation. Confocal images were taken using an Olympus FV1000 confocal microscope (Olympus, Center Valley, Pa., USA).

Viability Assay

FCSCs were seeded into a 96-well plate at a density of 2×10⁴ cells/well (100 μl) and treated with endotoxin-free PBS (vehicle control) or SBMSC-Exo (10⁸, 10⁹, or 10¹⁰ vesicles/ml) in either serum-free medium or CM containing 2% exosome-depleted FBS for a chemotaxis assay (see section 2.10) or ROS detection (see section 2.11), respectively. Cell viability was measured using a CellTiter 96® AQueous One Solution Cell Proliferation Assay (Promega, Madison, Wis., USA) according to the manufacturer's protocol. In brief, 20 μl of staining reagent was added in each well and incubated for 1 hour. The absorbance was read at 490 nm.

Chemotaxis Assay

Chemotactic migration was tested using a Transwell plate with a 8.0 μm-pore polycarbonate membrane insert (Corning). In brief, 1×10⁴ FCSCs were seeded on the top of the insert, and SBMSC-Exo suspended in serum-free CM was added in the reservoir. After 48 hours, non-migrated cells on the top of the insert were carefully removed by a cotton swab, and migrated cells were washed in PBS followed by the fixation in 4% buffered neutral formalin for 10 min. Then cells were stained with Richardson's reagent for 10 min and the number of positive cells were counted under the microscope.

ROS Detection

To investigate the ROS accumulation, FCSCs were seeded into 16-chamber slides at a density of 5×10³ cells/well (100 μl) and incubated in CM for 48 hours. FCSCs were treated with 150 μM H₂O₂ for 3 hours followed by the pre-treatment of SBMSC-Exo in CM containing 2% exosome-depleted FBS for 24 hours. This concentration and duration of H₂O₂ treatment was chosen because it induced significant ROS accumulation while maintaining cell viability. Cell viability and ROS accumulation were evaluated after staining with either 1 μg/mL Calcein AM and 10 μM Dihydroethidium (DHE; Thermo Fisher Scientific) or 10 μM 5-(and 6)-Carboxy-2′,7′-dichlorodihydrofluorescein diacetate (Carboxy-H₂DCFDA; Invitrogen, Carlsbad, Calif., USA) for 40 min. Green (Calcein AM and Carboxy-H₂DCFDA) and red (DHE) fluorescence were visualized by the confocal microscope. The number of positive DHE over Calcein AM was calculated from confocal images, and the ratio was normalized by the mean of positive control (H₂O₂⁺/SBMSC-Exo⁻).

ROS accumulation was also quantified by fluorescence-based microplate assays. FCSCs were seeded into a 96-well plate at a density of 1×10⁴ cells/well (100 μl) and incubated in CM containing 2% exosome-depleted FBS for 48 hours. FCSCs were treated with 150 μM H₂O₂ for 3 hours followed by the pre-treatment of SBMSC-Exo for 24 hours. Cells were washed with HBSS twice and stained with DHE or Carboxy-H₂DCFDA for 30 min in a 37° C. incubator. Fluorescence was measured at 518/606 and 495/529 (excitation/emission) for DHE and Carboxy-H₂DCFDA, respectively.

Statistical Analyses

Statistical analyses were performed using SPSS software (version 26; IBM, Armonk, N.Y., USA). Groups were compared by one-way analysis of variance (ANOVA) with the Tukey post hoc test. A p-value of less than 0.05 was considered significant.

### Results

Subchondral Bone-Derived Mesenchymal Stem Cells Release the Vesicles which Satisfy the Characteristic Criteria of the Exosomes.

SBMSCs obtained from the subchondral bone of young male bovine knees exhibited a long and flattened fibroblast-like morphology with a spindle cell body, and no morphological change was observed at passage 2 and 3. SBMSC-Exo fractions (Fractions 7-9) obtained from the SEC column had consistent yield with the mean of 13×10⁹ vesicles/ml (0.9−1.8×10⁹ vesicles/ml) (FIG. 1B). The size of SBMSC-Exo was between 60-150 nm with a peak of 60-70 nm from MRPS analysis (<0.5% standard error) (FIG. 1C). TEM images showed a similar range of size distribution (40-150 nm), and SBMSC-Exo showed cup-shaped morphology without any protein contamination (FIG. 1D). In immunoblotting array, SBMSC-Exo was positive for five putative exosome positive markers (FLOT-1, ICAM, CD81, ANXAS, and TSG101) whereas no cellular contamination protein (GM130) was detected (FIG. 1E).

FCSCs Migrates Toward the Impacted Lesion in Ex Vivo Fibrocartilage Culture

Seven days after the impact, migratory FCSCs with fibroblast-like morphology were observed on the edge of impacted area (FIGS. 2B and C). On the other hand, the cells in non-impacted area showed a rounded morphology. This implies that FCSCs may have a migratory ability toward the lesion although it is limited and needs to be improved.

SBMSC-Exo Internalizes into FCSCs and Induces Chemotactic Migration of FCSCs In Vitro

The PKH-67-stained SBMSC-Exo was internalized into FCSCs (green) and accumulated in the cytoplasm surrounding the nucleus of FCSCs (FIGS. 2E and G), which was in contrast to the control group where PKH-67 reagent in PBS was completely removed by a column filter (FIGS. 2D and F). The results of the cytotoxicity assay demonstrated that, under serum-free CM condition, the viability of FCSCs was maintained for 48 hours at SBMSC-Exo concentration range of 1×10⁸-10¹⁰ vesicles/ml (FIG. 2H). The results of the transwell migration assay showed that the number of migrated cells was significantly greater at 48 hours by 5.5 times (*p<0.05) in 1×10⁹ vesicles/ml of SBMSC-Exo group and by 14.8 times (**p<0.01) in 1×10¹⁰ vesicles/ml of SBMSC-Exo group than for the control group (FIG. 2I).

SBMSC-Exo Protects FCSCs Against H₂O₂ Induced Oxidative Damage.

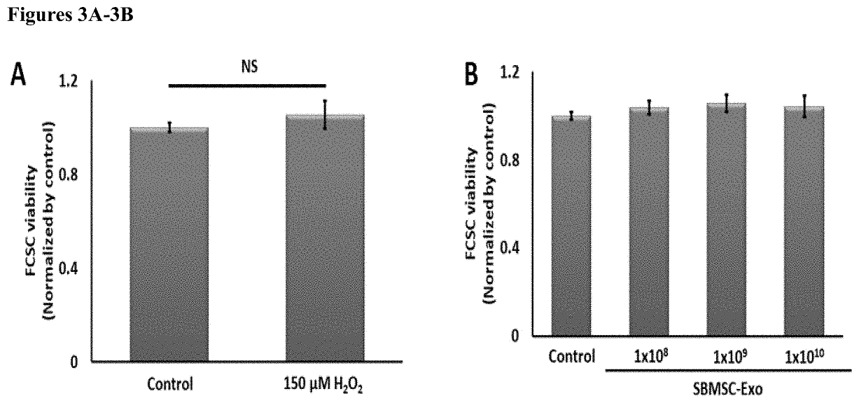

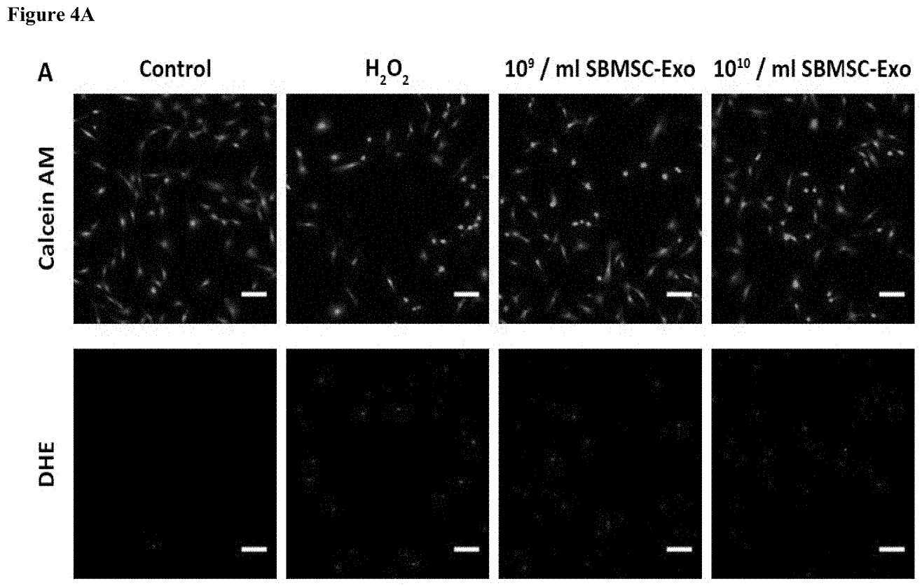

The results of the cytotoxicity test indicated that the treatment of H₂O₂ (150 μM for 3 hours) did not affect the FCSC viability (FIG. 3A). Likewise, no cytotoxic effect for SBMSC-Exo at a concentration range of 1×10⁸-10¹⁰ vesicles/ml was observed in FCSCs under the same CM condition (CM containing 2% exosome-depleted FBS) of H₂O₂ treatment at 24 hours (FIG. 3B).

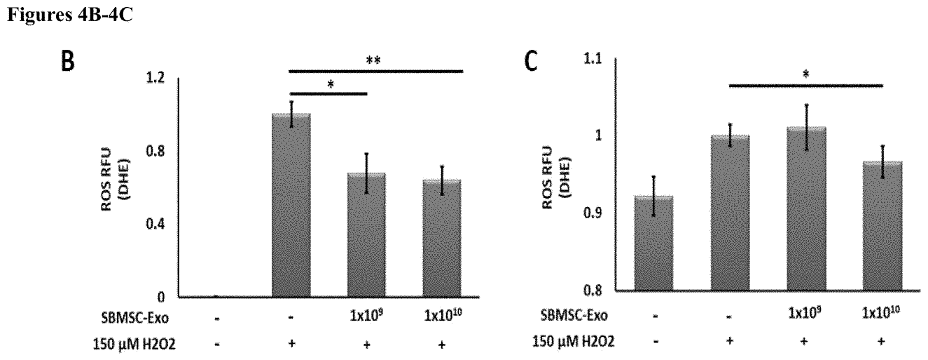

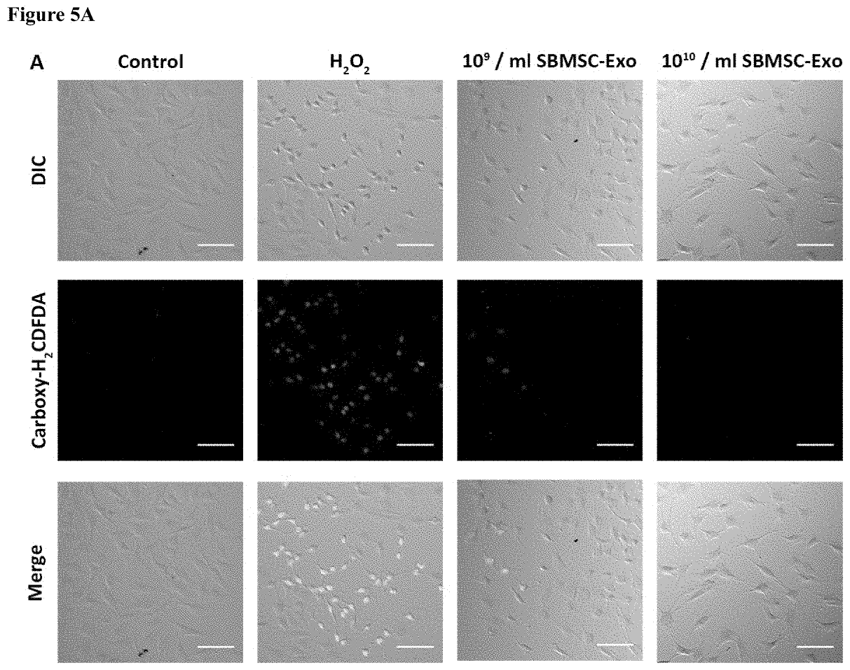

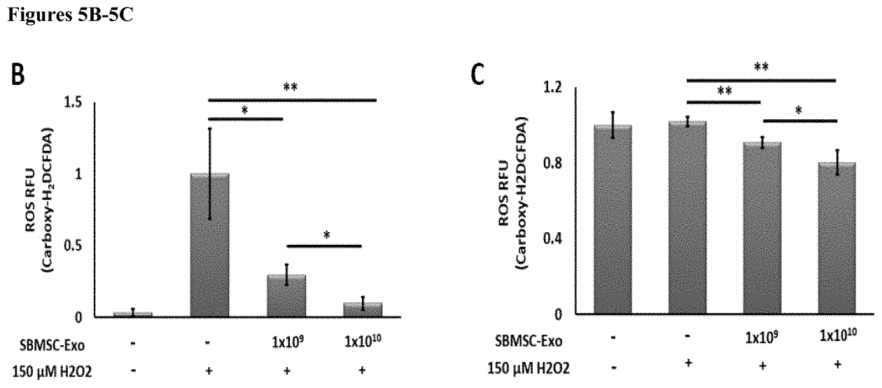

Although H₂O₂ did not induce the apoptosis of FCSCs, the morphology of FCSC was dramatically changed insofar as they lost the spindle and became round-shaped cells after the H₂O₂ treatment (H₂O₂⁺/SBMSC-Exo⁻) (FIGS. 4A and 5A). Those changes were rescued by the treatment of SBMSC-Exo (H₂O₂⁺/SBMSC-Exo⁺), especially in the higher dosage group of SBMSC-Exo (1×10¹⁰ vesicles/ml). Likewise, DHE and Carboxy-H₂DCFDA fluorescence was more intense in the group of H₂O₂⁺/SBMSC-Exo, which indicates that H₂O₂ induced ROS accumulation in FCSCs. The treatment of SBMSC-Exo (H₂O₂⁺/SBMSC-Exo⁺) significantly reduced the number of positive cells up to 37% DHE and 90% Carboxy-H₂DCFDA in a dose-dependent manner (n=3, *p<0.05, **p<0.01) (FIGS. 4B and 5B). The results of the microplate assay showed a similar phenomenon in that the ROS accumulation was significantly decreased by up to 4% of DHE and 20% of Carboxy-H₂DCFDA by the pre-treatment of SBMSC-Exo (H₂O₂⁺/SBMSC-Exo⁺) at a higher dosage (1×10¹⁰ vesicles/ml) with a statistical significance compared to the group of H₂O₂⁺/SBMSC-Exo⁻ (*p<0.05 and **p<0.01). In contrast, there was no significance at lower doses (1×10⁹ vesicles/ml) of SBMSC-Exo compared to the group of H₂O₂⁺/SBMSC-Exo⁻ (n=5) (FIGS. 4C and 5C).

### Discussion

Although MSCs derived from various sources, such as bone marrow, adipose, and umbilical cord, have been used for cell-based therapeutics due to their multi-differentiation potentials, increasing evidence implies that MSC-derived exosomes, which contain variety of functional cargoes including proteins, lipids, and nucleic acids, may play a crucial role in modulating the regenerative process for damaged tissue. For instance, MSC-derived exosomes enhanced bone and cartilage regeneration by increasing antioxidant ability, migration, proliferation, and differentiation. In addition, MSC-derived exosomes attenuated pain and inflammation and promoted matrix synthesis in the TMJ, which implies the possibility of interaction between MSC-derived exosomes and FCSCs in the TMJ. Although MSC-derived exosomes may have various therapeutic potentials, and especially, antioxidant and chemotaxis are crucial steps in the regenerative process for a damaged TMJ, those have not been extensively studied yet.

It was studied whether SBMSC-Exo promote FCSC chemotaxis and enhance antioxidant defenses, activities that may be of therapeutic value in the treatment of TMD. It is widely known that the superficial fibrocartilage layer of the mandibular condyles stores a reservoir of FCSCs, and fibrocartilage repair can be achieved by preserving and harnessing the regenerative potential of endogenous FCSCs. Meanwhile, unidirectional transfer of cytosolic proteins- and miRNA-loaded exosomes from MSCs to recipient cells improves their therapeutic and physiological functions. In this regard, throughout this study, the therapeutic and protective effects of SBMSC-Exo in FCSCs were studied.

Seven days after the impact injury in the fibrocartilage layer of bovine mandibular condyle, we observed the ex vivo migration of FCSCs toward the lesion. In addition, we observed that SBMSC-Exo stimulates chemotactic migration of FCSCs after 48 hours of the treatment in an in vitro experiment. Those observations imply that SBMSC-Exo would be a strong chemoattractant, and FCSC homing can be achieved by responding via a chemokine gradient provided by SBMSC-Exo. It is mainly consistent with the idea suggested in previous studies that the stem cell homing can be activated and facilitated by the expression of various cytokines and chemokines, such as CXCR4, CXCR7 and stromal cell-derived factor-1 (SDF-1), thereby delivering those factors via the exosome resulted in the enhanced migratory capacity of recipient cells by activating G-protein-mediated signaling pathways.

It was also confirmed that SMBSC-Exo helps protect FCSCs against oxidative damage. As expected, it was observed that the H₂O₂ increased the levels of DHE and Carboxy-H₂DCFDA in FCSCs, which indicated that ROS were over produced. It was also found that ROS accumulation was alleviated by SBMSC-Exo, which mirrors the outcomes shown in many previous publications. Others have suggested that, unlike the plasma-derived exosomes, MSC-derived exosomes alleviated the cellular aging in human cells by delivering peroxiredoxins, antioxidant enzymes to the recipient cells. Likewise, it was previously demonstrated that the transportation of MSC-derived exosome replenished depleted glycogenic enzymes including peroxiredoxins and glutathione S-transferases and reduce oxidative stress. Furthermore, MSC-derived exosomes also regulated mitochondrial health and inhibited the mitochondrial-induced apoptosis in rabbit chondrocyte via p38, ERK, and Akt pathways. Although many previous studies have focused on the significant role of the oxidative stress in the pathogenesis of TMD, it is also known that the migration of stem/progenitor cells can be interrupted by oxidative stress. For instance, induced pluripotent stem cells downregulate the expression of cell adhesion-related molecules in response to H₂O₂-induced oxidative stress, and their migration ability was also decreased. In this regard, the present results demonstrated that by delivering SBMSC-Exo, thereby enhancing various antioxidant mechanisms, mandibular fibrocartilage can be protected against oxidative damage while maintaining the regenerative ability of FCSCs.

### Conclusions

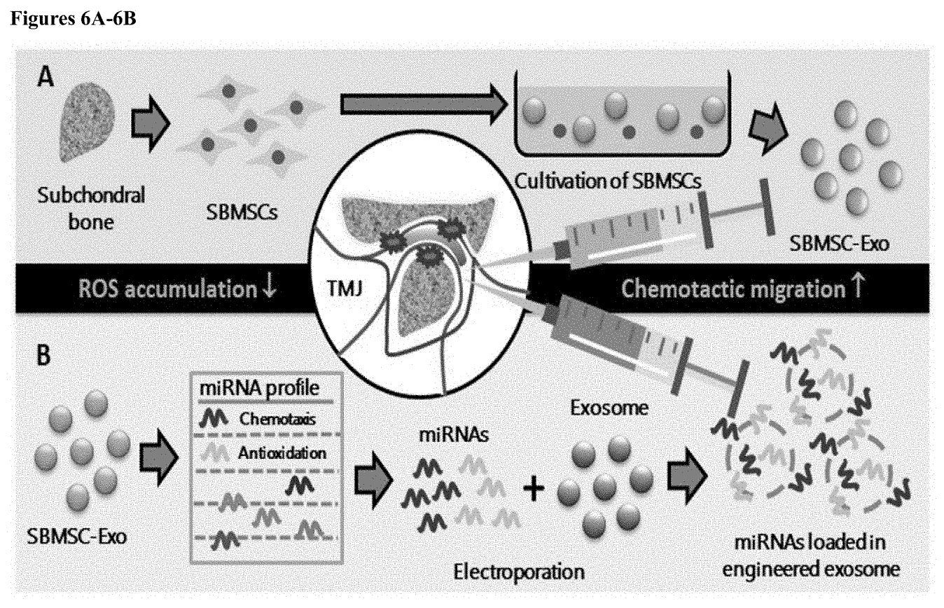

Collectively, it was demonstrated that SBMSC-Exo, the signaling molecules released from the mesenchymal stem cells, increases the capacity of FCSCs isolated from the superficial layer in the TMJ mandibular condyle to undergo migration, and it also protects FCSCs against H₂O₂-induced ROS accumulation (FIG. 6). We also discovered that highly purified SBMSC-Exo can be isolated from the culture medium by a SEC column. The overall outcomes of this study indicate that SBMSC-Exo would play a significant role in alleviating TMJ damage and promoting TMJ repair.

### Example 3

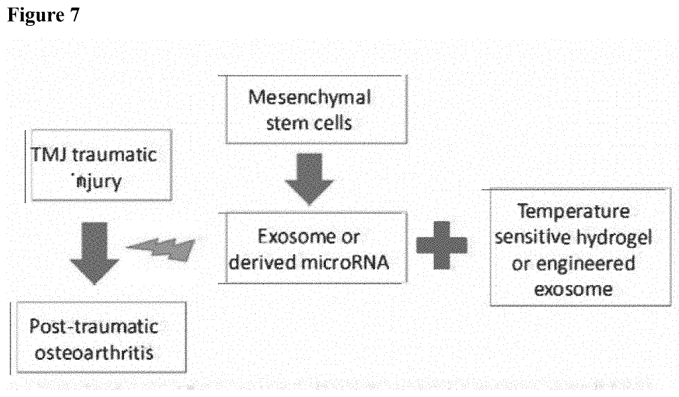

The goal of the research was to investigate the therapeutic potential of bone marrow stem cell-derived exosomes (BMSC-Exo) in post-traumatic osteoarthritis (PTOA) in temporomandibular joint (TMJ) (FIG. 7). Fibrocartilage stem cell (FCSC) was isolated from the bovine TMJ and was treated with BMSC-Exo. The results indicated that the exosomes enhanced FCSC migration and proliferation while alleviating the hydrogen peroxide-induced oxidative stress, which imply the clinical benefits of the exosomes for TMJ PTOA.

BACKGROUND: TMJ PTOA is critical TMJ disease, which is closely related to the fibrocartilage stem cell (FCSC)-regulated homeostasis of TMJ. Meanwhile, the exosomes, which deliver various cargo reflecting the status of parental cells, thereby, stem cell-derived exosomes mimicking the therapeutic potential of the stem cell therapy. In this regard, this study evaluated the antioxidant and regenerative effects of BMSC-Exo on FCSC, which play a major role in repair and regeneration of the damaged mandibular fibrocartilage.

MATERIALS AND METHODS: The BMSCs and FCSCs were collected from the male bovine femur and the mandibular condyle, respectively. BMSC-Exo were characterized in terms of the size distribution, zeta potential, morphology, exosomal marker proteins, and uptake by FCSC. FCSC migration and proliferation were investigated by chemotaxis and MTS assays, respectively. Oxidative stress was induced by hydrogen peroxide, and reactive oxygen species (ROS) levels were measured MitoSox and MitoTracker Green.

Results

BMSC-Exo Isolation and Characterization.

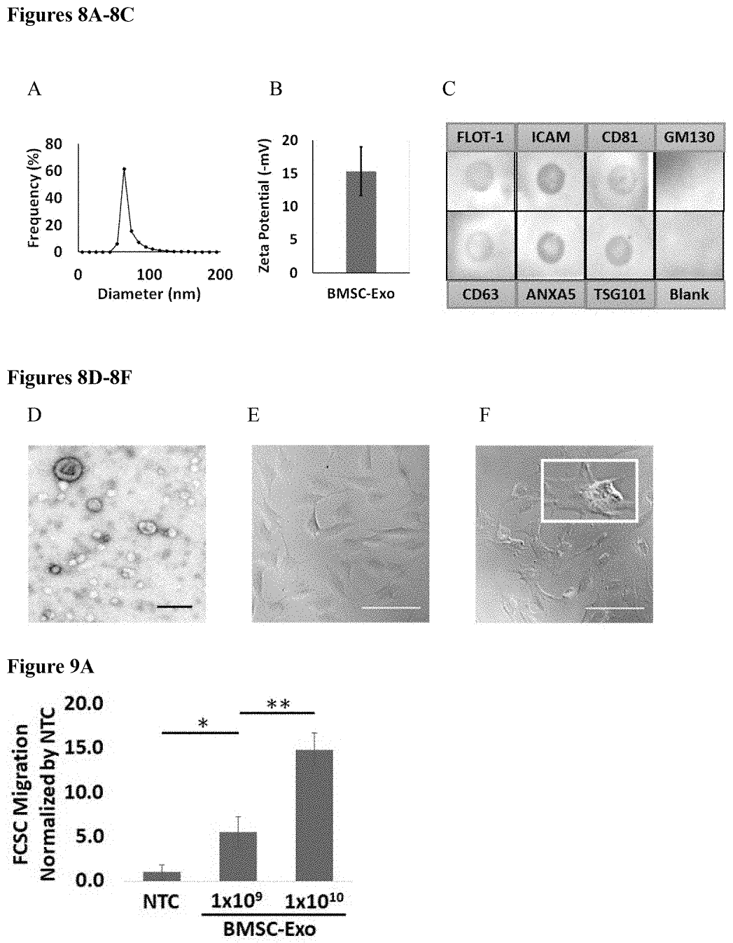

The concentration of BMSC-Exo was 4.36×10⁹/ml, and the size of BMSC-Exo was between 60-150 nm with a peak of 60-70 nm from microfluidic resistive pulse sensing analysis (FIG. 8A). Zeta-potential, which indicates the electric potential at the surface of BMSC-Exo, was −15.3 mV (FIG. 8B). BMSC-Exo was positive for six-putative exosome positive markers (FLOT-1, ICAM, CD81, CD63, ANAX5, and TSG101) whereas no cellular contamination protein (GM130) was detected (FIG. 8C). Transmission electron microscope (TEM) images showed a similar range of size distribution (40-150 nm), and BMSC-Exo showed cup-shaped morphology without any protein contamination (FIG. 8D). The internalization of BMSC-Exo into FCSC was observed from PKH67 fluorescent staining (FIGS. 8E and 8F).

Effect of BMSC-Exo on FCSC Proliferation and Chemotaxis.

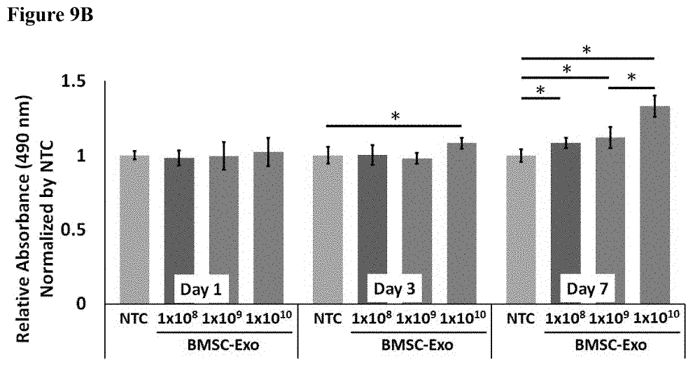

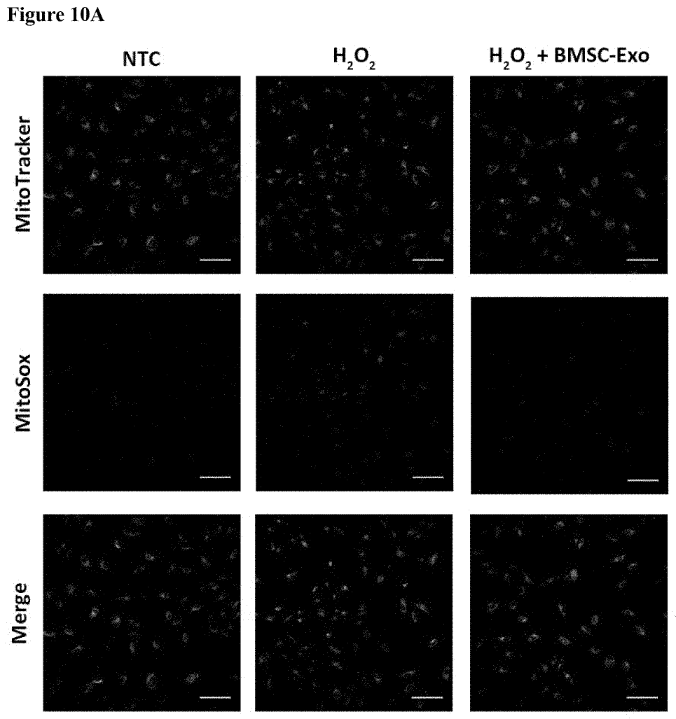

BMSC-Exo induced chemotactic migration of FCSC by 5.5-14.8 times at 48 hours in a dose-dependent manner (FIG. 9A). BMSC-Exo enhanced FCSC proliferation up to 33% in a time- and dose-dependent manner (FIG. 9B).

Antioxidant Effect of BMSC-Exo on FCSC.

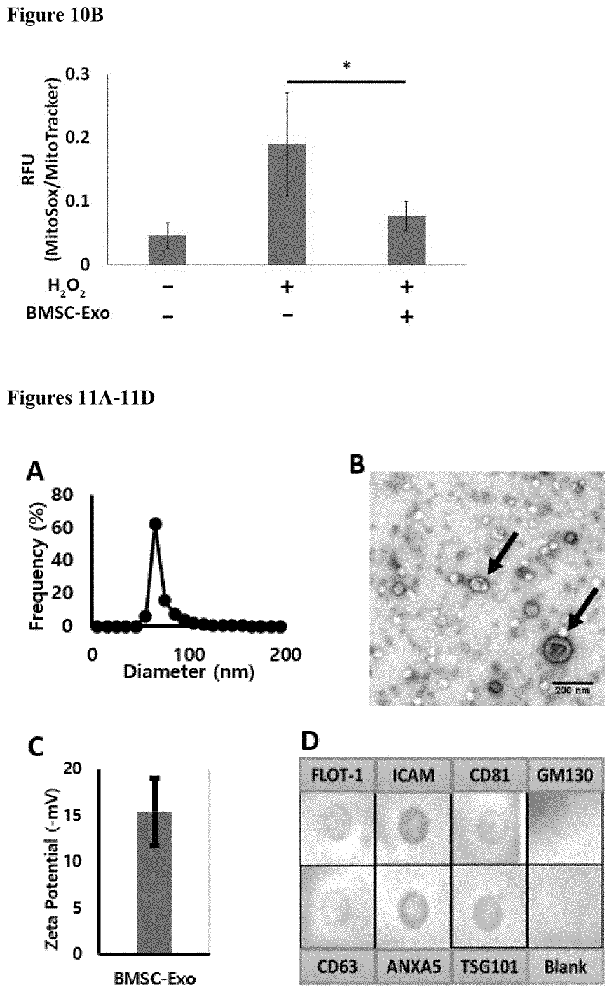

The level of mitochondrial superoxide, which indicates the oxidative stress of FCSC, was upregulated by the hydrogen peroxide (150 μM, 3 hours) and measured by MitoSox and MitoTracker dyes (FIGS. 10A-10B). The accumulation of mitochondrial superoxide was alleviated by BMSC-Exo by 60% (FIGS. 10A-10B).

SUMMARY: The exosomes can be isolated from size-exclusion chromatography. BMSC-Exo enhances FCSC proliferation and induces chemotactic migration of FCSC in a dose dependent manner. Hydrogen peroxide induces oxidative stress and upregulates the mitochondrial superoxide level in FCSC. BMSC-Exo protects FCSC from the accumulation of reactive oxygen species, which induce the oxidative damage in FCSC.

CONCLUSIONS: BMSC-Exo can play a pivotal role as chemoattractant and antioxidant, which enhances the regenerative capability of FCSC as well as protecting them from the oxidative stress; thereby, the application of BMSC-Exo in damaged TMJ mandibular condyle has potential to prevent the progression of TMJ PTOA while enhancing joint repair.

### Example 4

Bone Marrow Stem Cell-Derived Exosome: A Cell-Free Therapy for TMJ Repair

INTRODUCTION: Exosomes are extracellular vesicles, which are released from various eukaryotic and prokaryotic cells. Their major function is cell-to-cell communication via delivering various biomolecules. Especially, the contents of the exosomes reflect the status of parental cells, thereby, stem cell-derived exosomes deliver various cargo mimicking the therapeutic potential of the stem cell therapy while avoiding the risks that stem cell therapy may induce. Meanwhile, temporomandibular disorders (TMD) are the second most prevalent musculoskeletal conditions after chronic low back pain, and mandibular cartilage is the most frequently affected region by TMD. However, the lack of blood supply limits the regenerative capacity of the mandibular cartilage. Recently, many studies have suggested that the fibrocartilage stem cells (FCSC) reside underneath the superficial layer of the mandibular cartilage, and they play a critical role in the development and regeneration of the cartilage. In this regard, we propose the various therapeutic effects of the bone marrow stem cell-derived exosomes (BMSC-Exo) on FCSC for TMJ regeneration.

METHODS: Bovine bone marrow stem cells and the fibrocartilage stem cells were obtained from the subchondral region of the femur and superficial layer of the mandibular condyle, respectively. BMSC-Exo were obtained from BMSC-culture medium and purified by using size exclusion chromatography. Then, BMSC-Exo was characterized by TEM, MRPS, immunoblotting, Zetasizer methods. The proliferation and the chemotactic migration of FCSC were measured by MTS assay and Transwell assay, respectively. The oxidative stress was induced by the hydrogen peroxide, and ROS level was measured by a confocal microscope stained with MitoSox.

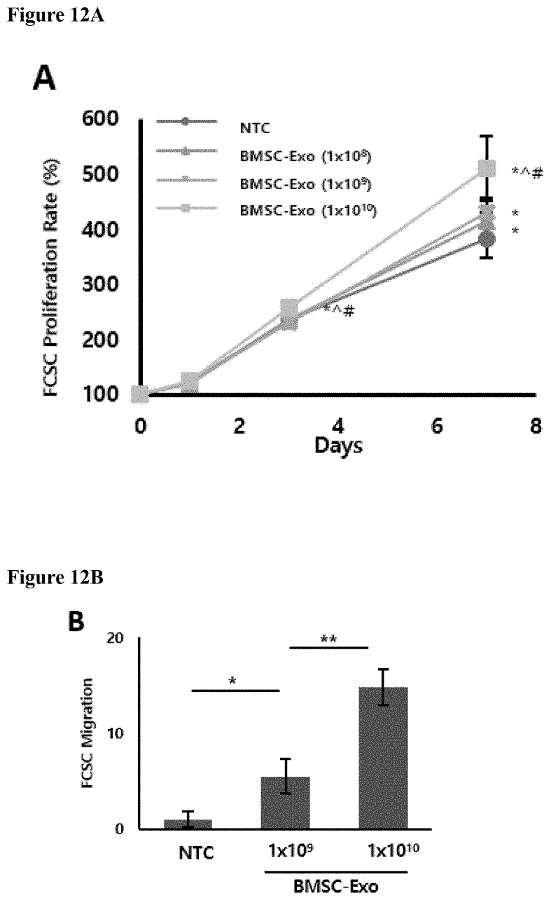

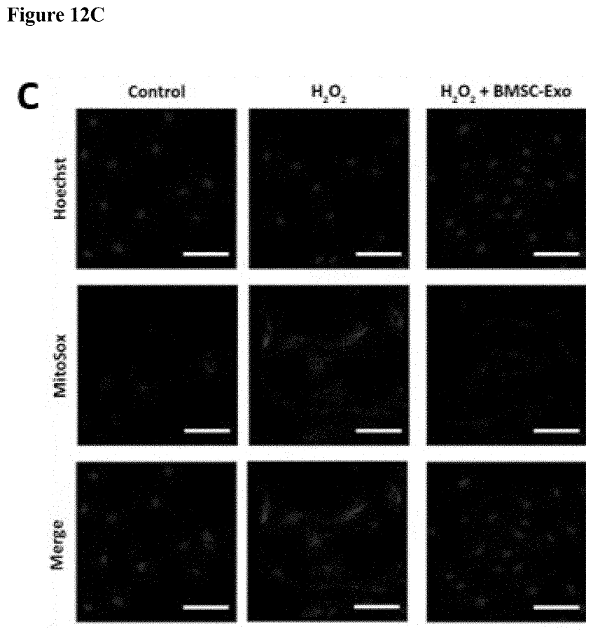

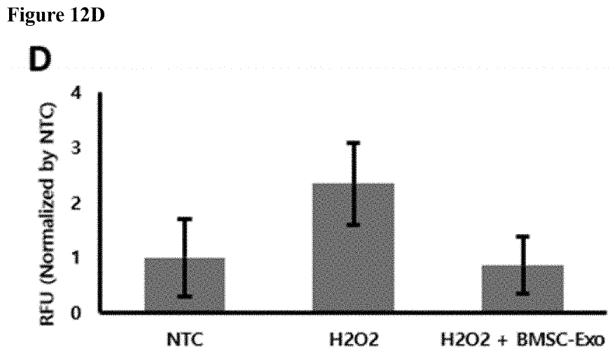

RESULTS: BMSC-Exo showed cup-shaped morphology and was sized between 50-150 nm. They were positive for putative exosome positive markers whereas no cellular contamination protein (GM130) was detected. The surface potential was −15.3 mV. MTS assay results showed that FCSC proliferation was increased by BMSC-Exo in a dose- and time-dependent manner (for 7 days, up to 33%). Likewise, the results of the Transwell migration assay showed that the number of migrated cells was significantly greater at 48 hours by 5.5 times (*p<0.05) in 1×10⁹ vesicles/ml of the BMSC-Exo group and by 14.8 times (**p<0.01) in 1×10¹⁰ vesicles/ml of BMSC-Exo group than for the control group. Lastly, the level of mitochondrial superoxide in FCSC was increased by 130% when they were treated with hydrogen peroxide, and it was alleviated by the treatment of BMSC-Exo. FIGS. 11A-11D show the characteristic of BMSC-Exo. FIGS. 12A-12D show the therapeutic effects of BMSC-Exo.

DISCUSSION: The result indicates that the biological cargo delivered by BMSC-Exo includes various advantageous substances, which play a key role in FCSC recruitment toward lesion by chemotaxis and the proliferation. Likewise, BMSC-Exo include potent antioxidants, thereby, the recipient cells (FCSC) can be protected against oxidative stress-induced damage. In conclusion, BMSC-Exo provide various therapeutic effects to the FCSC, which may enhance mandibular cartilage regeneration.

SIGNIFICANCE: This study elucidates the various beneficial biological effects of BMSC-Exo in degenerative diseases of TMJ. Those effects are closely related to the mandibular cartilage regeneration while preventing the progression of TMJ arthritis.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and “or” and similar referents in the context of describing the invention are to be construed to cover both the singular and the plural, unless otherwise indicated herein or clearly contradicted by context. Thus, for example, reference to “a subject polypeptide” includes a plurality of such polypeptides and reference to “the agent” includes reference to one or more agents and equivalents thereof known to those skilled in the art, and so forth.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

What is claimed is:

1. A method for preventing or treating post-traumatic osteoarthritis (PTOA) in the temporomandibular joint (TMJ) in a subject in need thereof, comprising administrating a pharmaceutical composition comprising an effective amount of mesenchymal stem cell-derived exosomes or mesenchymal stem cell-derived exosomal microRNA to the subject.

2. The method of claim 1, wherein the pharmaceutical composition is a local delivery system.

3. The method of claim 2, wherein the local delivery system comprises an injectable temperature-sensitive hydrogel.

4. The method of claim 2, wherein the local delivery system comprises an engineering exosome vehicle.

5. The method of claim 1, wherein the pharmaceutical composition is administered by injection to the subject.

6. The method of claim 1, wherein the pharmaceutical composition comprises an effective amount of mesenchymal stem cell-derived exosomes.

7. The method of claim 1, wherein the pharmaceutical composition comprises an effective amount of mesenchymal stem cell-derived exosomal microRNA.

8. The method of claim 1, wherein the pharmaceutical composition comprises bone marrow stem cell-derived exosomes (BMSC-Exo).

9. A method for preventing progressive fibrocartilage degeneration in a subject in need thereof, comprising administrating a pharmaceutical composition comprising an effective amount of mesenchymal stem cell-derived exosomes or mesenchymal stem cell-derived exosomal microRNA to the subject.

10. The method of claim 9, wherein the pharmaceutical composition is a local delivery system.

11. The method of claim 10, wherein the local delivery system comprises an injectable temperature-sensitive hydrogel.

12. The method of claim 10, wherein the local delivery system comprises an engineering exosome vehicle.

13. The method of claim 9, wherein the pharmaceutical composition is administered by injection to the subject.

14. The method of claim 9, wherein the pharmaceutical composition comprises an effective amount of mesenchymal stem cell-derived exosomes.

15. The method of claim 9, wherein the pharmaceutical composition comprises an effective amount of mesenchymal stem cell-derived exosomal microRNA.

16. The method of claim 9, wherein the pharmaceutical composition comprises bone marrow stem cell-derived exosomes (BMSC-Exo).
